(19) United States
(12) Patent Application Publication
(10) Pub. No.: US 2023/0338417 A1
(43) Pub. Date: Oct. 26, 2023

(54) Improved Pharmaceutical Preparation

(71) Applicant: CYTOPHARMA LTD., England (GB)

(72) Inventors: Mark Andre Freyberg, Freiburg (DE); Dirk Kaiser, Bullau (DE)

(21) Appl. No.: 18/025,700

(22) PCT Filed: Sep. 7, 2021
(86) PCT No.: PCT/EP2021/074603

§ 371 Date: Mar. 10, 2023

(30) Foreign Application Priority Data
Sep. 15, 2020 (DE) 10 2020 123 989.0

(51) Int. Cl. A61K 33/20; A61K 9/00; A61P 31/14

(52) U.S. Cl. CPC A61K 33/20; A61K 9/0078; A61P 31/14

(57) ABSTRACT

The present invention describes a pharmaceutical preparation in which a reactive chlorine-oxygen compound is provided in the form of a medicament inhalable into the lungs.

The present invention relates to an improved pharmaceutical preparation.

Pharmaceutical preparations containing a reactive chlorine-oxygen compound as active ingredient are known in the prior art and already show good properties as pharmaceuticals. However, there is an enduring need to improve these properties. These include, among others, the efficacy, tolerability, handling, dosage and dispensability of these pharmaceutical preparations.

In view of the prior art, it is therefore a task of the present invention to provide an improved pharmaceutical preparation. In this context, the pharmaceutical preparation should be producible as simply and inexpensively as possible. In particular, it should be possible to produce the pharmaceutical preparation in a large quantity. Furthermore, the pharmaceutical preparation should be very well tolerated and have as few side effects as possible. Furthermore, the pharmaceutical preparation should have a high efficacy. In particular, it should be possible to administer the pharmaceutical preparation already at the first signs of a condition which makes a medicinal treatment appear to be appropriate, without unacceptable harm to the patient being associated therewith. In addition, the pharmaceutical preparation should have such a high efficacy that even patients with a severe form of a disease state experience a significant increase in the probability of survival after administration of the drug.

Furthermore, the provision of a pharmaceutical preparation that can be handled safely and easily was a task of the present invention.

Furthermore, the pharmaceutical preparation should be easily adaptable to specifications with respect to dosage, so that, for example, a relatively high dosage can be applied. Furthermore, it was thus a task of the present invention to provide a pharmaceutical preparation which has excellent tolerability, in particular low side effects. Furthermore, the pharmaceutical preparation should contribute to an improvement of the quality of life and not additionally impair it.

Furthermore, the pharmaceutical preparation to be provided should lead to a strong improvement in the survival rate of patients with a severe disease, especially a severe lung disease or a more severe infectious disease, especially a bacterial or viral infectious disease.

These tasks, as well as other tasks not explicitly mentioned, but which are readily derivable or inferable from the interrelationships discussed herein, are solved by a pharmaceutical preparation having all the features of claim 1.

Thus, the subject-matter of the present invention is to provide a pharmaceutical preparation containing, as an active ingredient, a reactive chlorine-oxygen compound, which is characterized in that the reactive chlorine-oxygen compound is provided in the form of a medicament inhalable into the lungs.

In particular, the present invention has the effect of unexpectedly improving the efficacy, tolerability, manageability, dosage and dispensability of a pharmaceutical preparation containing a reactive chlorine-oxygen compound as an active ingredient.

In particular, a pharmaceutical preparation according to the present invention enables a surprisingly effective treatment of a lung disease. In this regard, a reliable treatment with few side effects can be achieved in patients with a lung disease, which, among other things, improves the course of the disease and reduces the severity of the disease. In particular, the survival rate of patients with severe lung diseases, some of which are associated with a high mortality rate, can be greatly increased.

Furthermore, a pharmaceutical preparation according to the present invention shows excellent tolerability, in particular low side effects. Furthermore, a pharmaceutical preparation according to the present invention leads to an improvement of the quality of life and does not additionally impair it.

Furthermore, a pharmaceutical preparation according to the invention is very well tolerated and exhibits relatively low side effects. On the other hand, a pharmaceutical preparation according to the present invention exhibits a high efficacy, whereby the pharmaceutical preparation can be administered already at the first signs of a disease, preferably an infectious disease and/or a pulmonary disease, particularly preferably a pulmonary infectious disease, without unacceptable harm to the patient being associated therewith. Moreover, a pharmaceutical preparation according to the present invention exhibits such a high efficacy that even patients with a severe form of an infectious disease and/or a pulmonary disease, particularly preferably a pulmonary infectious disease, experience a significant increase in the probability of survival after administration of the drug.

Here, the pharmaceutical preparation is preferably relatively simple and inexpensive to produce. Furthermore, a pharmaceutical preparation can be produced in a large quantity.

Furthermore, the pharmaceutical preparation can be handled safely and easily. Particularly surprisingly, the drug can be applied not only by professional caregivers but also directly by untrained persons, for example patients, so that, for example, self-care of an infectious disease and/or a pulmonary disease, particularly preferably a pulmonary infectious disease, is enabled.

Furthermore, the pharmaceutical preparation can be easily adapted to specifications in terms of dosage, so that, for example, relatively high or low dosages can be applied so that this can be adapted to the respective needs of the patients.

The pharmaceutical preparation containing as active ingredient a reactive chlorine-oxygen compound is provided in the form of a medicament inhalable into the lungs. The term “lung inhalable” means that the medicament can penetrate to the lungs. Preferably, the reactive chlorine-oxygen compound is provided as an aerosol.

Preferably, it can be provided that the reactive chlorine-oxygen compound is provided as an aerosol with an average particle size of at most 10 μm, preferably at most 5 μm, more preferably at most 3.5 μm, measured as Mass Median Aerodynamic Diameter (MMAD) with a 7-stage cascade impactor (Next Generation Impactor (NGI)) (DIN EN 13544-1:2007+A1:2009).

Furthermore, it can be provided that the reactive chlorine-oxygen compound is provided as an aerosol, wherein the mass fraction of particles with a particle size smaller than 5 μm is at least 50%, preferably at least 60% and particularly preferably at least 70%, measured with a 7-step cascade impactor (Next Generation Impactor (NGI)).

The pharmaceutical preparation of the present invention comprises, as an active ingredient, at least one reactive chlorine-oxygen compound. Here, the pharmaceutical preparation of the present invention may comprise exactly one, two, three, four or more reactive chlorine-oxygen compounds. Reactive chlorine-oxygen compounds are compounds comprising oxygen and chlorine that contribute to an improvement of a disease state, illness or disease. These include lung diseases and/or infectious diseases, such as bacterial or viral infectious diseases, as detailed previously and hereinafter. Further, these include, but are not limited to, chronic lung diseases. Here it can be assumed that reactive chlorine-oxygen compounds may chemically or biochemically interfere with the processes described above and below that contribute to a disease, without this being intended to be limiting

Preferably, oxygen acids of chlorine, their salts or derivatives of these oxygen acids can be especially used as reactive chlorine-oxygen compounds. Derivatives of reactive chlorine-oxygen compounds include carbonic acid adducts of oxygen acids of chlorine or similar compounds.

Reactive chlorine-oxygen compounds are already used or at least proposed for some pharmaceutical applications. Thus hypochlorites, in particular sodium hypochlorite, are used in dentistry or for treating patients with atopic eczema. Chlorites, including sodium chlorite, are used for treating wounds, wherein its use for the treatment of amyotrophic lateral sclerosis (ALS) is further proposed. Chlorates, in particular sodium chlorate, are used for treating wounds, and perchlorates, in particular potassium perchlorate, are used for treating thyroid disorders. However, these compounds have not yet been made available in the form of a drug that can be inhaled into the lungs.

Furthermore, peroxochlorine compounds are known from WO 00/48940 A1 and dichlorine compounds from WO 2005/049483 A2, which are particularly suitable as agents for treating wounds. Provision of these compounds in the form of a drug that can be inhaled into the lungs has not been proposed to date.

Surprisingly, it has been established that the peroxochlorine compounds described in WO 00/48940 A1 and the dichlorine-oxygen compounds known from WO 2005/049483 A2 can be provided in the form of a medicament inhalable into the lungs, thereby achieving unexpected improvements.

Document WO 00/48940 A1, filed at the European Patent Office on 18.02.2000 with application number PCT/EP00/01350, in particular the peroxochloric acids, their derivatives and anions and methods of production described therein and the pharmaceutical preparations described therein are mentioned here for disclosure purposes by reference to this publication. Document WO 2005/049483 A2, filed at the European Patent Office on 22.11.2004 with application number PCT/EP2004/013212, in particular the dichloric acids disclosed therein, derivatives, anions and salts thereof as well as methods for their production and the pharmaceutical preparations described therein are mentioned here for disclosure purposes by reference to this publication

Here the dichloroxo acids presented in WO 2005/049483 A2 are superior to the peroxochloric acids described in WO 00/48940 A1. This applies in particular to their efficacy and their compatibility as well as their shelf life. Thus dichloroxo acids are particularly preferred. This also applies to the salts, anions and derivatives of these acids.

Peroxochloric acids or the salts thereof, which are described inter alia in WO 00/48940 A1, and dichloroxo acids or the salts thereof, which are described inter alia in WO 2005/049483 A2, are superior to hypochlorites, chlorites, chlorates and perchlorates. Thus peroxochloric acids and dichloroxo acids acids are preferred, wherein dichloroxo acids are particularly preferred. This applies in particular to their efficacy and tolerability. This also applies to the salts, anions and derivatives of these acids (peroxochloric acids and dichloroxo acids).

Preferably, it may be provided that the reactive chlorine-oxygen compound comprises a peroxochloric acid, a peroxochlorous acid and/or a dichloroxo acids, preferably a dichloroperoxo acid or a pharmaceutically tolerable salt of these acids. Furthermore, preferred reactive chlorine compounds include preferably dichloroxo acids, particularly preferably dichloroperoxo acids, their intermediates such as peroxochloric acid and peroxochlorous acid and their respective derivatives, salts and anions. Here, dichloroxo acids are preferred, wherein dichloroperoxo acids are preferred over other reactive chlorine-oxygen compounds. Dichloroxo acids are oxyacids of chlorine, which have two chlorine atoms. Dichloroperoxo acids are oxyacids of chlorine, which have two chlorine atoms and at least four, preferably six oxygen atoms.

In a particular embodiment, it may be provided that the reactive chlorine-oxygen compound comprises a molecular formula selected from HClO, HClO₂, HClO₃, HClO₄ and/or H₂Cl₂O₆ or a pharmaceutically acceptable salt of these acids, preferably selected from HClO₃, HClO₄ and/or H₂Cl₂O₆ or a pharmaceutically acceptable salt of these acids.

It may preferably be provided, that the reactive chlorine-oxygen compound comprises a structure of formula [O═ClOO]⁻, [O₂ClOO]⁻, [O₂ClOOClO₂]²⁻ and/or the anion of the reactive chlorine compound comprises the molecular formula Cl₂O₆²⁻. This structure may be present here as an acid and/or salt, wherein the salt is preferably pharmaceutically acceptable.

Preferably, peroxochloric acids are used with the molecular formula HClO₄, wherein the anion preferably has a structure [O₂ClOO]⁻, which in particular has a peroxo group (O—O). In the case of a preferably used peroxochloric acid, chlorine has the oxidation state of +5, wherein these compounds are described inter alia in WO 00/48940 A1.

Preferably, a reactive chlorine-oxygen compound, preferably a peroxochloric acid or a salt of this acid, is obtainable by a method in which
(a) chlorine dioxide is reacted with an aqueous or water-containing solution of hydrogen peroxide or another hydroperoxide or peroxide at a pH>=6.5,
(b) the pH is lowered to 3 to 6 by the addition of an acid,
(c) the gaseous free reactive chlorine compound is expelled with a cooled gas and collected in a basic solution with a pH>10.

Further preferred embodiments of this method are presented with reference to the dichloric acids described below and which are used particularly preferably. Here the methods differ in particular regarding step d). Steps a) to c) apply accordingly.

Particularly preferably, dichloroxo acids are used with the formula H₂Cl₂O₆ and their derivatives, anions, or salts as reactive chlorine-oxygen compounds. These compounds are also referred to here as dichloroperoxo acids, regardless of the structure of the anion of this acid.

The exact structure of the reactive chlorine-oxygen compound, in particular of the dichloroperoxo acids is not essential here, wherein the following structures can be assumed. In particular dichlorine oxygen acids are preferred with the formula H₂Cl₂O₆ and their derivatives, anions, or salts, with the structural formulae of the anions

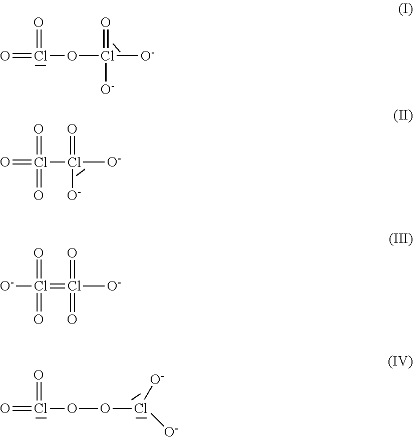

wherein dichloric acids of the anions of the structural formulae I-III are particularly preferred.

Preferred dichloroxo acid to be used are shown in the following Table 1. Of these dichloroxo acid, dichloroxo acids no. 1 to no. 3 represent particularly preferred embodiments of the compounds to be used.

TABLE 1

| | Formal oxidation | | |
|---|---|---|---|
| No. | states of chlorine | Structural formula of the acid | Structural formula of the Di-anion |
| 1 | +5, +5 | 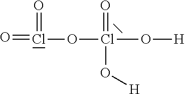 | 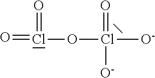 |
| 2 | +6, +4 | 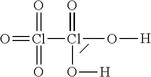 | 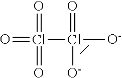 |
| 3 | +5, +5 | 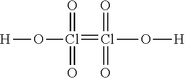 | 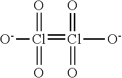 |
| 4 | +5, +3 | 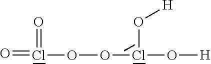 | 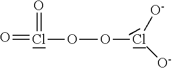 |

In addition to the valances already described +3/+5 (WO 00/48940) and +4/+4 (Bogdanchikov G. A., Kozlov, Y. N. and Berdnikov, V. M. “The Mechanism of the Elementary Act of HO₂-Anion Oxidation by a ClO₂ Radical in Aqueous Solution” Khim.Fiz. 1983 (5), 628-636) the dichloric acids no. 1 to no. 3 used according to the invention with the valences of +6/+4 and +5/+5 for chlorine can also and preferably be used for the production of a pharmaceutical preparation according to the invention.

The reaction of peroxochlorate ions O₂ClOO⁻ with chlorite ions (ClO₂⁻) leads directly to the range of “dimer” Cl₂O₆²⁻ species, which can preferably be used:

O_{c}ClOO⁻+ClO₂⁻→Cl₂O₆²⁻->->and isomers

Furthermore, surprisingly the peroxochlorite ion, O═ClOO⁻ and peroxochlorous acid O═ClOOH derived therefrom can be used for the production of pharmaceutical preparations according to the invention.

Particularly around the neutral point, the decomposition of the dichloric species Cl₂O₆²⁻ into chlorate ions ClO₃⁻ and peroxochlorite ions OClOO⁻ is in clear competition with the described intramolecular redox reactions of the dichloric species, leading to compounds 1-4 in the above Table.

When reference is made to anions in the present disclosure, the presence of required counter ions is included (mainly in solution). The term anions is primarily used to express that in solution the dichlorate (Cl₂O₆²⁻) is the more stable form compared to the protonated acid (H₂Cl₂O₆). However, according to the invention, depending on the context, the term “anion” can also be representative of the acid, the term “acid” can also be representative of the “anion”. The counter ions preferably represent pharmaceutically acceptable cations, which are generally known.

The reactive chlorine-oxygen compounds to be used according to the invention can also be used as a mixture. Thus, dichloroxo acids and peroxochlorous acid and also the anion present at physiological pH values can therefore be present in solution as a mixture with peroxochlorate and chlorite according to the invention and can be used as such. Such a solution comprising the dichloroxo acids, peroxochlorous acid, peroxochlorate and chlorite is therefore one of the particularly preferred exemplary embodiments of the present invention.

However, since large amounts of chlorite are detrimental to the use of dichloroxo acids in the pharmaceutical sector, it is particularly preferred if chlorite is present in the end product of the solutions according to the invention in an excess amount of no more than 20 times, preferably no more than 5 times, and in particular no more than 3 times by weight over other reactive chlorine compounds, in particular dichloroxo acids, preferably dichloroperoxo acids, relative to the total weight of the solution.

In particular, the preferred dichloroxo acids to be used and the peroxochlorous acid are present in this solution in amounts of about 0.1-20 wt. %, preferably 3-5 wt. %, based on the weight of ClO₂ used. The qualitative detection is achieved by Raman spectroscopy. Performing this type of spectroscopy is a matter of course for the person skilled in the art in this field. The obtained spectrograms of dichloroxo acids, preferably dichloroperoxo acids, are significantly different from the compositions which are obtained by the method of WO 00/48940. The quantitative portion can be determined by titration.

It has already been mentioned that dichloroxo acids, preferably dichloroperoxo acids are preferred over other reactive chlorine compounds, in particular also peroxochloric acid. Accordingly, it is particularly preferred, if peroxochlorate (ClO⁴⁻) is present in the end product of the solutions according to the invention in an excess amount of no more than 20 times, preferably no more than 5 times and in particular no more than 3 times by weight over other reactive chlorine-oxygen compounds, in particular dichloroxo acids, preferably dichloroperoxo acids, relative to the total weight of the solution. Particularly preferably, the proportion of peroxochlorate (ClO⁴⁻) is below 25 wt. % preferably below 15 wt .%, particularly preferably below 5 wt. % relative to the weight of the dichloroxo acids contained.

Further qualitative detection is possible by the reaction with the haem iron. In the presence of preferred dichloroxo acids, preferably dichloroperoxo acids, the time curve of the change in intensity of the Soret band is markedly different from that of the solutions obtained by the method of WO00/48940.

Particularly preferably, a reactive chlorine-oxygen compound is obtainable according to a method, in which
(a) chlorine dioxide is reacted with an aqueous or water-containing solution of hydrogen peroxide or another hydroperoxide or peroxide at a pH>=6.5,
(b) the pH is lowered to 3 to 6 by the addition of an acid,
(c) the gaseous free reactive chlorine compound is expelled with a cooled gas and collected in a basic solution with a pH>10, and
(d) the collected reactive chlorine compound is incubated with chlorite at a pH of 6 to 8, preferably approximately 7.

By a method comprising steps (a) to (d) in particular dichloroxo acids, preferably dichloroperoxo acids are obtained.

The reactive chlorine-oxygen compounds to be preferably used can be obtained in particular by a method which preferably consists of reacting chlorine dioxide with aqueous or water-containing hydrogen peroxide or another peroxide or hydroperoxide known to a person skilled in the art, e.g. peroxocarbonate or perborate or the urea adduct of hydrogen peroxide at a pH of 6.5 or higher, preferably pH 10-12. It is preferable to keep the pH at a constant value.

It should be noted here that peroxochloric acid and its anions and derivatives which occur as an intermediate, can also be obtained by reacting chlorine dioxide with other oxidizing agents which contain the peroxo grouping.

The reaction can be carried out in an aqueous medium or in a water-containing medium. For example, in addition to water, solvents miscible with water can be used, such as alcohols, e.g. alkanols, such as methanol, ethanol or the like, or mixtures thereof.

Optionally, other chloroxides can also be used. For example, chlorine monoxide, preferably in its dimeric form (Cl₂O₂), can also be reacted with a hydroperoxide (preferably hydrogen peroxide) to obtain the desired product. The reaction works in the same pH range as for chlorine dioxide.

The reaction temperature can be increased, for example up to about 50° C.; in purely aqueous systems the lowest temperature is preferably about 0° C. However, chlorine dioxide should not be used below +10 degrees Celsius, since below this temperature the chlorine dioxide gas liquefies and deflagrations may occur. If there are additional organic solvents and/or high concentrations of the reagents involved, lower temperatures can also be used, i.e. below the freezing point of water. Preferably, this is conducted at room temperature.

The chlorine dioxide required for the reaction is available to the person skilled in the art and can be produced in the usual way. For example, it can be produced by reacting a chlorite with an acid (for example sodium chlorite with sulfuric acid) or by the reduction of chlorate, for example with sulfuric acid.

The chlorine dioxide obtained in this way can be released if necessary after the removal of any traces of chlorine in a known manner (Granstrom, Marvin L.; and Lee, G. Fred, J. Amer. Water Works Assoc. 50, 1453-1466 (1958)).

If the chlorite used for the production of ClO₂ is contaminated with carbonate, this produces ClO₂ contaminated with CO₂, and/or the carbonic acid adducts described in WO00/48940. For the absorption of carbon dioxide, the gas stream containing chlorine dioxide and carbon dioxide should be passed through a wash bottle charged with lye. If contact times are short, the CO₂, but not the ClO₂ will be absorbed by the lye. However, it is better to free the carbonate impurities by fractional crystallization of the sodium chlorite used. Any contamination of the peroxochlorate with carbonate can be easily identified in the Raman spectrum. Instead of the sharp band at 1051 cm⁻¹ a double band is obtained at 1069 cm⁻¹ (broad) and the band at 1051 cm⁻¹ (sharp).

The chlorine dioxide can be carried with an inert gas, such as nitrogen or a noble gas such as argon, but also by air or oxygen to react with the peroxo compound or hydroperoxide, such as the hydrogen peroxide or percarbonate or perborate. For example, it is possible to produce the chlorine dioxide in a first reaction vessel and introduce it with the said gases or a mixture thereof into a second reaction vessel in which the peroxo compound (peroxide or hydroperoxide) is in an aqueous or water-containing solution.

The pH of the reaction mixture is kept equal to or above 6.5 by the addition of a base. It is preferable to keep the pH constant. This can be done either manually or automatically by a “pH-stat” device.

Common inorganic or organic bases, such as alkaline lyes, for example sodium hydroxide or potassium hydroxide, earth alkali hydroxides, ammonia or organic bases such as nitrogen bases, can be used as the bases. The hydroxides of quaternary ammonium salts, in particular alkyl, such as trialkyl or tetraalkyl ammonium hydroxides or zinc hydroxides can also be used.

The content of hydroperoxide in the reaction mixture can be determined for example by potentiometric titration with an acid, such as for example hydrochloric acid.

The solutions obtained according to the method described above can be used as such or also in a modified form. For example, excess hydrogen peroxide can be removed in the usual way, e.g. with a heavy metal compound such as manganese dioxide. Excess amounts of other oxidizing agents can be removed in a similar way.

Excess chlorine dioxide (ClO₂) can be removed with H₂O₂. This should be done as soon as possible, as otherwise by

2 ClO₂+2OH⁻->ClO₂⁻+ClO₃⁻+H₂O

the disruptive ClO₃⁻ with pentavalent chlorine (chlorate) would form. However, a product containing chlorate is not preferred over other pharmaceutical preparations, so that the formation of chlorate should be avoided. Particularly preferably, the proportion of chlorate (ClO₃⁻) is below 25 wt. %, preferably below 15 wt .%, particularly preferably below 5 wt. %, based on the weight of the oxychloric acids contained.

To improve the shelf life of the reaction product, for example storage at an elevated pH value is suitable, for example at a pH of 10 or more. This pH value can be adjusted with a suitable base, as described above for the method of production.

To produce solutions, which contain the preferred dichloroxo acids and/or peroxochlorous acid and/or the salts of these said acids to be preferably used, it has been made possible surprisingly to expel and capture the free peroxochlorous acid HOOClO, the dichloroxo acids or the peroxochloric acid when the pH is below 6, e.g. a pH 5 or less from the mixture containing the obtained chlorite ions, with an inert gas, such as a noble gas, e.g. argon or nitrogen or also the gases oxygen or air, particularly preferably the free peroxochlorous acid HOOClO, the dichloroxo acids and the peroxochloric acid are expelled by an oxygen-rich gas. Surprisingly, it has been shown that the yield can be increased considerably if the gas path is kept very short and the gas flow is cooled.

The mixture formed during the introduction in step (a) of the production process described above initially contains very high concentrations of chlorite ions (ClO₂⁻). The content of chlorite can however be significantly reduced by “passing” it in the gas stream into a basic solution. Here, the chlorine-oxygen acids of all kinds are expelled as volatile compounds in protonated (neutral) form, which are very unstable however. A base is present in the sample, whereby the chlorine acids are depronated and anions are formed. After adjusting the solution to pH 6-8 and after adding defined amounts of chlorite, for example in the form of sodium chlorite, the anions of the more preferred dichloroxo acids are formed.

Preferred dichloroxo acids are obtainable by a method in which the reactive chlorine-oxygen compound obtained according to steps a) to c) described above is incubated with chlorite at a pH of 6 to 8. The incubation time can be selected more or less at random, wherein incubation times that are too short lead to incomplete conversion, and incubation times that are too long lead to possible decomposition of the more preferred dichloroxo acids. Preferably, the incubation time can be in the range of 1 second to 1 week, particularly preferably in the range of 1 minute to 24 hours and especially preferably 5 minutes to 1 hour. The incubation time can be controlled by adjusting the pH value, wherein an increase of the pH value to above 8, in particular above 9, ends the incubation.

The amount of chlorite can be within a broad range. Large amounts of chlorite in relation to the reactive chlorine compound obtained according to steps a) to c) described above lead to a very complete conversion to the particularly preferred dichloric acids. Small amounts of chlorite in relation to the reactive chlorine compound obtained according to steps a) to c) described above lead to residual amounts of the reactive chlorine-oxygen compounds obtained according to steps a) to c) described above.

Here, the chlorite can be used in an excess amount of up to 100 times, preferably up to 10 times, with respect to the reactive chlorine oxygen compound obtained according to the aforementioned steps a) to c). In a particularly preferred embodiment, it can be provided that the molar ratio of chlorite to the reactive chlorine oxygen compound obtained according to the aforementioned steps a) to c) is in the range of 10:1 to 1:10, preferably 2:1 to 1:2 and more preferably 1:1 to 1:1.2.

As on the one hand large excess amounts of chlorite should be avoided and on the other hand the dichloroxo acids obtainable by incubation with chlorite are preferable to other reactive chlorine compounds, preferably equimolar amounts of chlorite and of the reactive chlorine oxygen compound obtained according to steps a) to c) described above are used.

In step c), the gaseous free reactive chlorine compound can be collected, for example, in a base, such as an alkali metal base, alkaline earth metal or zinc base or nitrogen base, such as ammonia or an organic amine. However, it is also possible to freeze out the gaseous acids in a cold trap (e.g. at −100 to −190 ° C.).

All metal cations and organic cations, such as those of nitrogen bases, in particular quaternary ammonium salts, can be used as counter ions. For pharmaceutical applications, alkaline earth metals or alkali metals are preferred in particular, preferably Na⁺ or K⁺, or Zn²⁺.

It is expedient and preferred to store the acids according to the invention and their derivatives, salts and/or anions with the exclusion of light and to produce aqueous solutions from them with high pH values, e.g. with pH values of 10, 11 or 12 and above, in particular in the range of pH 10 to pH 13, in order to achieve long-term storage capabilities. From such solutions, the free acid can be recovered as required, as described above, and if necessary converted into solutions with a desired pH or into salts.

Preferably, the pharmaceutical preparation shows a signal in a mass spectrum at 189.0 m/z. This signal is preferably based on the reactive chlorine-oxygen compound used. Accordingly, reactive chlorine-oxygen compounds are preferred, which show a signal in the mass spectrum at 189.0 m/z.

Furthermore, the mass spectrum of the pharmaceutical preparation may have a signal at 99 m/z, wherein the signal is preferably based on the reactive chlorine-oxygen compound used.

In a preferred embodiment of the pharmaceutical preparation or the reactive chlorine-oxygen compound, it may be provided that the signal at 189.0 m/z of the mass spectrum of the pharmaceutical preparation is higher than at 99 m/z.

Furthermore, the mass spectrum of the pharmaceutical preparation may have a signal at 83.2 m/z, wherein the signal is preferably based on the reactive chlorine-oxygen compound used.

In a preferred embodiment of the pharmaceutical preparation or the reactive chlorine-oxygen compound, it may be provided that the signal at 83.2 m/z of the mass spectrum of the pharmaceutical preparation is higher than at 99 m/z.

The mass spectrum can be obtained by conventional methods, wherein it is preferably performed according to the method given in the examples.

Furthermore, a preferred pharmaceutical preparation for use according to the present invention may have a reactive chlorine-oxygen compound which, in an ion chromatogram, shows a peak at a retention time of 15 min. The ion chromatography can be performed using conventional methods, wherein this is preferably performed according to the method given in the examples.

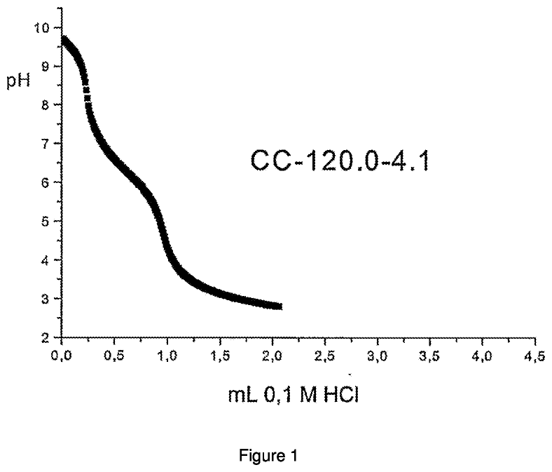

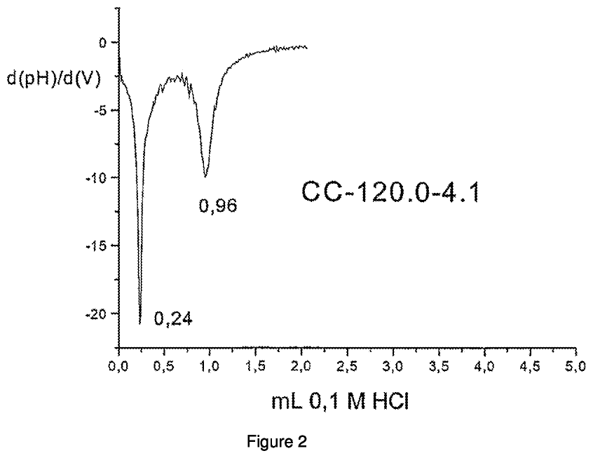

The particularly preferred dichloroxo acids preferably have two transitions in a titration curve, as shown in FIGS. 1 and 2. This titration curve shows two transitions, which can be assumed to be caused by the pKa values of the particularly preferred dichloroxo acids. Accordingly, the particularly preferred dichloroxo acids preferably have two pKa values, wherein one pKa value is in the region of 8.5 and one pKa value is in the region of 5. These values are determined potentiometrically at 25° C. with a 0.1 M hydrochloric acid.

For comparison, pKa values of further reactive chlorine-oxygen compounds are listed below:

| | | | | Acid |
|---|---|---|---|---|
| | Name of the | Formula | | dissociation |
| Oxidation | acid and | of the | | constant as |
| number | its salts | acid | Anion | pKa at 298K⁾ |
| +I | hypochlorous | HOCl | hypochlorite | 7.25 |
| | acid | | OCl⁻ | |
| | hypochlorites | | | |
| +III | chlorous acid | HOClO | chlorite | 2 |
| | chlorites | | ClO₂⁻ | |
| +V | chloric acid | HOClO₂ | chlorate | 0 |
| | chlorates | | ClO₃⁻ | |
| +VII | perchloric acid | HOClO₃ | perchlorate | −9 |
| | perchlorates | | ClO₄⁻ | |
| +V | peroxochloric | HOOClO₂ | peroxochlorate | approx. 6.2 |
| | acid | | —OOClO₂ | |

The dichloroxo acids, or the peroxochlorous acid, their respective derivatives, or anions and salts, which are preferred according to the invention, can be provided as such and, in particular, in aqueous or water-containing solution as a pharmaceutical preparation the form of a medicament inhalable into the lungs. In this form, unexpected advantages are obtained in the treatment of a variety of diseases, in particular in the treatment of pulmonary diseases.

The preparations can contain the active ingredient alone or preferably together with one or more pharmaceutically applicable carriers.

The pharmaceutical preparation can preferably comprise a pharmaceutically acceptable carrier. The pharmaceutically acceptable carrier is preferably adapted to the form of administration. Preferably, the pharmaceutical carrier comprises water, wherein the water content is preferably at least 90 wt. % based on the weight of the pharmaceutical carrier, preferably based on the weight of the pharmaceutical preparation. In a preferred embodiment, it can be provided that the pharmaceutical preparation is an aqueous solution. The aqueous solution here comprises the reactive chlorine-oxygen compound described above.

Preferably, it can be provided that the reactive chlorine-oxygen compound is present in the pharmaceutical preparation in a molar concentration of at least 0.01 mmol/l, preferably at least 0.1 mmol/l, particularly preferably at least 0.3 mmol/l and especially preferably at least 0.5 mmol/l, wherein preferably water is used as the pharmaceutically acceptable carrier and the amount of reactive chlorine-oxygen compound is determined by titration. The concentration of reactive chlorine-oxygen compound can be determined by any suitable method, wherein preferably titration is performed with HCl. The concentration of HCl in the titration solution is preferably in the range of 0.01 to 1 mold, particularly preferably about 0.1 mold. Here, the concentration of reactive chlorine compound is determined by the pH value of the composition. Further details on this can be taken from the examples.

Preferably, it can be provided that the reactive chlorine-oxygen compound is present in the pharmaceutical preparation in a molar concentration in the range of 0.01 mmol/l to 100 mmol/l, preferably 0.1 mmol/l to 50 mmol/l, particularly preferably 0.3 to 20 mmol/l and especially preferably from 0.5 mmol/l to 10 mmol/l, wherein water is preferably used as the pharmaceutically acceptable carrier and the proportion of reactive chlorine compound is determined by titration. The molar concentration can preferably be determined by titration with HCl, as explained in more detail above.

Preferably, it can be provided that the reactive chlorine-oxygen compound is present in the pharmaceutical preparation in a concentration by weight of at least 2 mg/l, preferably at least 20 mg/l, particularly preferably at least 60 mg/l and especially preferably at least 100 mg/l, wherein preferably water is used as the pharmaceutically acceptable carrier and the amount of reactive chlorine compound is determined by titration. The concentration of reactive chlorine-oxygen compound can be determined by any suitable method, wherein preferably titration is performed with HCl, as described above. The molar mass of the reactive chlorine-oxygen compound is used for the conversion, wherein in a preferred embodiment, in particular in the case of dichloroxo acids with a molecular formula Cl₂O₆²⁻ this can be taken to be about 167 g/mol. Preferably, the anion can be used as the basis.

Preferably, it can be provided that the reactive chlorine-oxygen compound is present in the pharmaceutical preparation in a molar concentration in the range of 2 mg/l to 20000 mg/l, preferably 20 mg/l to 10000 mg/l, particularly preferably 600 mg/l to 4000 mg/l and especially preferably 100 mg/l to 2000 mg/l, wherein water is preferably used as a pharmaceutically acceptable carrier and the amount of reactive chlorine-oxygen compound is determined by titration. The concentration of reactive chlorine compounds based on the weight can preferably be obtained by titration with HCl and a subsequent conversion, considering the molar mass, wherein particularly preferably the molar mass of the anion is used.

In addition to a reactive chlorine-oxygen compound a pharmaceutical preparation may comprise at least one further active ingredient, which is different from a reactive chlorine-oxygen compound, as this is the subject-matter of the present application. A further subject-matter of the present invention is a pharmaceutical preparation having at least one reactive chlorine-oxygen compound, as described above and in the following, and at least one pharmaceutically active substance, which differs from the reactive chlorine-oxygen compound given above and in the following.

Preferred further active ingredient other than a reactive chlorine-oxygen compound include, but are not limited to, steroids, such as cortisone; beta-2 sympathomimetics, such as indacaterol, salbutamol, fenoterol, reproterol, salmeterol, and formoterol; anticholinergics, such as butylscopolamine, ipratropium bromide, and tiotropium bromide.

The dosage of the reactive chlorine-oxygen compound to be used according to the invention can be selected according to the disease, the clinical picture and the condition of the patient. In one embodiment, the pharmaceutical preparation can be provided in a form which is administered in a single dose. Preferably, the pharmaceutical preparation is provided in the form of a preparation that can be administered multiple times. For example, it may be provided that the pharmaceutical preparation can be administered at least twice, preferably at least three times, particularly preferably at least four times and especially preferably at least five times.

Preferably, the pharmaceutical preparation is provided such that there is a period of 1 hour to 5 days, preferably 2 hours to 2 days and especially preferably 3 hours and 1 day between the administrations.

The dosage of the reactive chlorine-oxygen compound to be used according to the invention depends on the disease to be treated, the clinical picture to be treated, as a well as on the species, their age, weight and individual condition, individual pharmacokinetic conditions as well as the mode of application. Preferably, the dosage (preferably in humans) is in the range of 0.01 to 100 pmol/kg, in particular between 0.1 to 100 pmol, i.e. for example in a human with a body weight of 70 kg a 1 mg to 1 g/day, in particular 8.5 mg to 850 mg/day, in a single dose or divided into several doses.

The reactive chlorine-oxygen compound is provided in the form of a medicament inhalable into the lungs. Preferably, the pharmaceutical preparation is provided via an inhaler. In particular, it may be provided that the inhaler provides the pharmaceutical preparation with a nebulization time of at most 10 min for 2.5 ml of pharmaceutical preparation. Preferably, it may be provided that the inhaler provides the pharmaceutical preparation with a nebulization rate of 0.10 ml/min to 2 ml/min, preferably 0.15 to 1.0 ml/min and particularly preferably 0.20 ml/min to 0.60 ml/min.

Furthermore, it can be provided that the pharmaceutical preparation is provided with a volumetric flow rate in the range of 0.05 l/s to 10 l/s, preferably from 0.1 l/s to 4 l/s, particularly preferably 0.2 l/s to 3 l/s (liters per second).

The pharmaceutical preparation containing as active ingredient a reactive chlorine-oxygen compound is provided in the form of a medicament inhalable into the lungs. Accordingly, a reactive chlorine-oxygen compound is introduced into the lungs via a gas stream, preferably an air stream. Preferably, an amount of 0.5 ml to 15 ml, preferably 1 to 10 ml, more preferably 2 to 8 ml of pharmaceutical preparation can be provided per inhalation process over a period of 1 second to 2 hours, preferably 1 minute to 60 minutes, more preferably 10 minutes to 30 minutes. The amount of pharmaceutical preparation is calculated here on the liquid to be vaporized or the solid to be aerosolized, which preferably comprises at least one pharmaceutically acceptable carrier, preferably water, in addition to the reactive chlorine-oxygen compound.

It can further be provided that the pharmaceutical preparation is provided with a concentration in the range of 1*10⁻⁶ to 20% by volume, preferably in the range of 1*10⁻⁵ to 5% by volume, more preferably in the range of 0.0001 to 1% by volume, this indication referring to the ratio of the volume of the pharmaceutical preparation to nebulizing gas (air).

The dosage of further active ingredients, which are different from a reactive chlorine-oxygen compound, can be chosen according to the clinical picture to be treated, as well as species, their age, weight and individual condition, individual pharmacokinetic conditions, as well as the mode of application of the further active substance(s), whereby these can be chosen in accordance with the usual dose/doses for the further active substance(s).

The invention also relates to a pharmaceutical composition for the prophylactic and in particular therapeutic treatment of disease states described therein, preferably for prophylactic or therapeutic treatment, preferably of a warm-blooded animal suffering from such a disease, containing one or more reactive chlorine compounds, preferably peroxochloric acid, peroxochlorous acid and/or dichloric acids, particularly preferably dichloroxo acids and/or peroxochlorous acid, more preferably dichloroperoxo acids, or their respective derivatives or salts in an amount effective prophylactically or in particular therapeutically to combat said disease and one or more pharmaceutically applicable carriers in the form of a medicament inhalable into the lungs.

The invention also relates to a method for treating disease conditions, preferably for prophylactic and/or therapeutic treatment—in particular in a warm-blooded animal, in particular a human-, comprising the administration of reactive chlorine compounds, preferably peroxochloric acid, peroxochlorous acid and/or dichloric acids, particularly preferably dichloroxo acids and/or peroxochlorous acid, more preferably dichloroperoxo acids, or their respective derivatives, anions or salts in an amount effective against said diseases to a warm-blooded animal, e.g. humans, in need of such treatment in the form of a medicament inhalable into the lungs.

The invention also relates to the use of reactive chlorine compounds, preferably peroxochloric acid, peroxochlorous acid and/or dichloric acids, preferably dichloroxo acids and/or peroxochlorous acid, particularly preferably dichloroperoxo acids, and their derivatives, anions or salts for use in a method for treating the human or animal body in the form of a medicament inhalable into the lungs.

The invention relates in particular to the use of reactive chlorine compounds, preferably peroxochloric acid, peroxochlorous acid and/or dichloric acids, preferably dichloroxo acids and/or peroxochlorous acid, particularly preferably dichloroperoxo acids, their derivatives, anions or salts for the production of a medicament for the treatment of the human or animal body, preferably for the prophylactic and/or therapeutic treatment—in particular in a warm-blooded animal, in particular a human—of the disease states described above or below in the form of a medicament inhalable into the lungs.

The medicament inhalable into the lungs may be provided in appropriate unit dose forms. Preferably, the medicament inhalable into the lungs can be applied via an inhaler.

The inhalate can be supplied to the inhaler by conventional methods, with corresponding storage forms being known. For example, the medicament can be stored in the form of, for example, ampoules, vials, syringes or bags and supplied to the inhaler accordingly for application. Other forms of application, in particular for solutions of reactive chlorine compounds, preferably dichloric acids, preferably dichloroxo acids, particularly preferably dichloroperoxo acids or peroxochlorous acid, their ions, derivatives or salts, are, for example, sprays provided in a lung-inhalable form and the like. The unit dosage forms, e.g. ampoules, vials, syringes or bags, preferably contain from about 0.005 g to about 10.0 g, in particular from 8.5 mg to 850 mg, of a salt of reactive chlorine compounds, preferably peroxochloric acid, peroxochlorous acid and/or dichloric acids, particularly preferably dichloroxo acids and/or peroxochlorous acid, more preferably dichloroperoxo acids, their anions, derivatives with usual carriers.

It can further be provided that the pharmaceutical preparation is provided in a container comprising at least two chambers for storing at least two liquids, which chambers are openable by mechanical action so that after opening of the chambers the liquids are mixable, wherein one of the chambers comprises a liquid a reactive chlorine-oxygen compound of the present invention and one of the chambers comprises a liquid adjusted to a physiological pH value for adjusting the pH value. In particular, this container is used for storing the pharmaceutical preparation. In a particular embodiment of the present invention, the container is adapted to an inhaler in such a way that the container is inserted into the inhaler and the inhaler, when operated, opens the at least two chambers for storing at least two liquids and mixes the liquids. In a further step, the inhaler preferably provides the reactive chlorine-oxygen compound in the form of a medicament inhalable into the lungs.

This method of delivery is particularly useful for reactive chlorine-oxygen compounds, whose pH value has to be adjusted to a physiologically acceptable value. This applies, inter alia to the oxoacids of chlorine described above which have a good shelf life at a high pH. If further active ingredient, which is different from a reactive chlorine compound, is used with it, this can be stored in one of the two compartments, depending on its stability. Preferably however, this is provided in a third compartment, the container being designed such that firstly the pH of the reactive chlorine compound is adjusted to a physiologically acceptable value, after which the obtained mixture is mixed with the contents of the third compartment.

The pharmaceutical preparations of the present invention are produced in a known manner, e.g. by means of conventional mixing, dissolution, or lyophilization methods.

In a preferred embodiment, a 0.005 to 1 M solution of one or more reactive chlorine compounds, preferably peroxochloric acid, peroxochlorous acid and/or dichloric acids, particularly preferably dichloroxo acids and/or peroxochlorous acid, more preferably dichloroperoxo acids and/or a salt of these acids or their derivatives can be dissolved in double-distilled water at a pH equal to or >10, preferably 10 to 13, in particular 12.5. Directly before administration, this solution is diluted to isotonicity to concentrations of about 1-10 mM with common salt, sodium or potassium bicarbonate and double-distilled water and approximated to the physiological pH. This solution is suitable for application via the lungs. For this purpose, an inhaler can preferably be used.

For a preferred formulation of a medicament for inhalative use, the reactive chlorine compounds, preferably peroxochloric acid, peroxochlorous acid and/or dichloric acids, particularly preferably dichloroxo acids and/or peroxochlorous acid, very particularly preferably dichloroperoxo acids, or their derivatives as salts are dissolved in double-distilled water with concentrations in the lower millimolar or upper micromolar range, preferably in a concentration range of 0.5-10 mM with a pH equal to or >10, in particular 10 to 13, preferably e.g. pH 11.5 and adjusted to isotonicity with glycerine, common salt or another suitable compatible, preferably physiological agent. Before use, a physiological pH value is adjusted with a physiologically acceptable acid, preferably HCl. Further additives are possible. In particular, when filling the drug into plastic containers, additives are suitable which can neutralize traces of transition metals, since transition metals are dissolved into the walls during storage and can catalyze a decomposition of the active ingredient. Examples of such additives are oligo- or polyalcohols, such as ethylene glycol, desferrioxamine or EDTA (e.g. as disodium EDTA). The solution obtained in this way can be administered directly by inhalation, preferably via an inhaler, particularly preferably as an aerosol.

The anions of the preferably used dichloric acids or peroxochlorous acid are stable, the acids themselves decompose relatively quickly. The stabilization of the active pharmaceutical ingredient can therefore be achieved via the pH value. The active ingredient solution can be lowered to an almost physiological value by buffer dilution immediately before use to improve tolerability.

Since the dichloric acids or the peroxochlorous acid to be used are defined compounds, there should also be no difficulties regarding the approval of the drug.

The pharmaceutical preparation is used in particular for the treatment of infectious diseases or pulmonary diseases, preferably pulmonary diseases.

Preferably, it can be provided that the pharmaceutical preparation is provided for use in the treatment of a lung disease. Here, the lung disease may be a mycotic, bacterial or viral infection preferably a bacterial or viral infection, more preferably a viral infection.

It can further be provided that the bacterial lung disease is an infection caused by Streptococcus pneumoniae, in particular Pneumococcus, Pseudomonas aeruginosa, Burkholderia cepacia complex (BCC), Staphylococcus (S.) aureus, methicillin-resistant Staphylococcus aureus, Pandoraea (related to Burkholderia), Stenotrophomonas maltophilia, Achromobacter xylosoxidans, Haemophilus influenzae, Nocardia, Ralstonia pickettii, Inquilinus, Enterobacteria, and/or Mycobacteria, such as Mycobacterium tuberculosis, Mycobacterium bovis, Mycobacterium africanum Mycobacterium microti.

Surprisingly, reactive chlorine-oxygen acids administered via the lungs exhibit very high efficacy against bacteria that are particularly critical and difficult to treat. This is surprisingly true, among others, for Pseudomonas aeruginosa, Burkholderia cepacia complex (BCC), Staphylococcus (S.) aureus, methicillin-resistant Staphylococcus aureus, Mycobacterium tuberculosis, Mycobacterium bovis, Mycobacterium africanum Mycobacterium microti.

S. pneumoniae belong to the Gram-positive bacteria that can cause a variety of diseases. These diseases can be particularly dangerous to infants, young children, the elderly, and people with underlying chronic conditions.

Pseudomonas aeruginosa is the most common germ found in the respiratory tract in cystic fibrosis. Pseudomonads are found mainly in humid environments, especially in moist biotopes and stagnant water, but also in the lungs of humans.

They can be transmitted from person to person, which is why care must be taken in clinics to separate Pseudomonas aeruginosa-positive patients from other cystic fibrosis patients. Pseudomonas aeruginosa has the ability to form a biofilm in which the bacteria aggregate and, embedded in a protective gelatinous layer, survive the attacks of the immune system and antibiotics.

Early and acute infections with pseudomonads can be treated well with antibiotics. Therefore, patients with cystic fibrosis must be regularly screened for pseudomonads to detect and treat the infection early. Once the bacteria have taken up residence in their biofilm in the lungs, patients are usually afflicted by recurrent infections (chronic infection), which are increasingly difficult to treat due to the emergence of resistance and impair lung function in the long term.

Bacteria of the Burkholderia cepacia complex (BCC) are biofilm-forming moist germs and naturally insensitive to many antibiotics. They also readily acquire further resistance, which can result in multidrug-resistant forms. BCC bacteria are found in soil, especially in the root zone of certain plants. The BCC comprises a group of several genetically similar bacterial species. Bacteria of the complex occur much less frequently than pseudomonads in patients with cystic fibrosis, but the course of infection can be much more severe and is often more difficult to treat. However, this is not equally true for all species of BCC. Of particular significance for cystic fibrosis patients are Burkholderia (B.) multivorans, B. cenocepacia and B. dolosa. Under certain circumstances, these bacteria can trigger the so-called cepacia syndrome, an acute deterioration of lung function with life-threatening complications. After lung transplantation, B. gladioli and B. cenocepacia can lead to severe infections.

Staphylococci are spherical bacteria that occur in the environment and also naturally in healthy people on the skin and mucous membrane. Staphylococcus (S.) aureus is a very resistant germ and can survive for months even in dry dust or on surfaces.

S. aureus is the most common germ in young cystic fibrosis patients and is later usually “replaced” by other germs, especially Pseudomonas aeruginosa. Infection with S. aureus can proceed without symptoms, but in cystic fibrosis patients it can also lead to increased coughing with purulent sputum and, rather rarely, cause severe pneumonia.

Non-resistant S. aureus are also called methicillin-sensitive S. aureus (MSSA): They can usually be treated well with antibiotics. However, an infection with MSSA can also become chronic and impair lung function in the long term.

A special form of the bacterium Staphylococcus aureus is the methicillin-resistant form (MRSA). With these bacteria, almost all so-called beta-lactam antibiotics (penicillins, almost all cephalosporins and carbapenems) are ineffective. Only a few antibiotics are available for this germ and it is not always possible to combat it. This is particularly problematic because the risk of infection is high and this can make it difficult to deal with other patients.

The mycobacteria, especially Mycobacterium tuberculosis, Mycobacterium bovis, Mycobacterium africanum and/or Mycobacterium microti often cause tuberculosis, especially pulmonary tuberculosis.

It can further be provided that the viral lung disease is an infection caused by respiratory syncytial viruses, rhinoviruses, influenza viruses, parainfluenza viruses, human bocaviruses, corona viruses (e.g., the SARS and MERS viruses), adenoviruses, and/or human metapneumoviruses.

Surprisingly, reactive chlorine-oxygen acids administered via the lungs show very high efficacy against viruses that are particularly critical and difficult to combat. This is surprisingly true for corona viruses, in particular SARS-associated coronavirus, Middle East respiratory syndrome-related coronavirus, severe acute respiratory syndrome coronavirus 2 (SARS-CoV-2), respiratory syncytial viruses, rhinoviruses, influenza viruses, and others.

Coronaviruses cause very different diseases in humans, ranging up to severe acute respiratory syndrome. In this case, infections of the lower respiratory tract occur, especially in co-infections with other respiratory pathogens. Severe courses of the disease are observed especially in pre-existing diseases.

Respiratory syncytial virus infections often occur in infancy and early childhood and are the most common cause of respiratory infections at this age. Cystic fibrosis patients are more likely to show a more complicated course with RSV infection and poorer lung function for months afterward. In addition, RSV infection increases the risk for bacterial and other viral infections.

Rhinoviruses (“cold” or “common cold” viruses) mainly cause symptoms in the upper respiratory tract. However, in CF patients, they can also infect the deeper airways. In studies, CF patients with rhinovirus infection have been shown to require significantly longer courses of antibiotics, indicating more severe or prolonged bacterial infections of the lungs as a result of rhinovirus infection. However, long-term damage to the lungs by rhinoviruses has not been observed to date.

The influenza virus causes the “real” flu, which is different from flu-like infections with other viruses. Influenza virus infection can cause severe and long-term deterioration in the lung function of cystic fibrosis patients, and severe lung damage, including lung failure, can also occur. However, the infection usually progresses without long-term complications. Cystic fibrosis patients should therefore be vaccinated annually against influenza.

It can further be provided that the mycotic lung disease is an infection caused by Aspergillus fumigatus, Scedosporium, Exophiala dermatitidis and/or Candida.

Aspergillus fumigatus is a mold. Its spores, which are suspended in the air, can be inhaled and thus reach the deep respiratory tract of humans. This can trigger various symptoms, especially in immunocompromised individuals. For cystic fibrosis patients, colonization of the airways with Aspergillus can lead to allergic bronchopulmonary aspergillosis (ABPA). In ABPA, the patient's immune system reacts “allergically” to Aspergillus, i.e., it produces substances that cause excessive inflammation of the lungs. As a result, the airways narrow and breathing can be threateningly restricted.

The black mold Scedosporium is a rare pathogen of skin and soft tissue infections that can cause serious complications, especially after transplantation due to immunosuppression. In patients with cystic fibrosis, Scedosporium (S. aurantiacum, S. prolificans, S. apiospermum) can cause symptoms in the respiratory tract similar to ABPA (see above).

The mold Exophiala dermatitidis, closely related to Scedosporium, is also a rare pathogen that can invade the human body and cause skin and soft tissue infections. This fungus has also been found in cystic fibrosis patients and has been associated with pneumonia. After transplantation, very rare invasive infections with Exophiala can cause serious complications.

The yeast Candida (most commonly known as Candida albicans) is found in most people on the mucous membranes of the mouth, throat, genital area and digestive tract without causing symptoms there. The most common clinical picture is thrush, i.e. local infection of the mucous membranes with redness, whitish coating and itching. However, Candida can also lead to infections in the respiratory tract. Chronic colonization with Candida can worsen lung function in cystic fibrosis patients.

Further, the pharmaceutical preparation provided in the form of a medicament inhalable into the lungs may preferably be provided for use in the treatment of a chronic lung disease

Surprisingly, the present invention particularly provides a method of treating chronic lung diseases that are difficult to treat. In particular, it may be provided that the chronic lung disease is bronchial asthma and/or COPD (chronic obstructive pulmonary disease).

Bronchial asthma, often referred to simply as asthma, is a chronic, inflammatory disease of the airways. The inflammation can lead to seizure-like shortness of breath due to narrowing of the airways (bronchial obstruction). This results in increased mucus production, the bronchial muscles become tense, and formation of edema of the bronchial mucosa.

An asthma attack can last from a few seconds to several hours. In Germany, about ten percent of children and five percent of adults suffer from bronchial asthma. The airways of asthmatics react to certain, otherwise mostly harmless stimuli (e.g. mental stress, overexertion) with increasing sensitivity and constrict spasmodically. Triggers can also be
allergens,
respiratory infections,
coldness,
medicinal products and
polluted air.

Asthma can be diagnosed based on medical history, physical examination, and with the help of pulmonary function tests and allergy testing. Treatment for allergic asthma consists of avoiding the allergen.

In the case of an acute asthma attack, an asthma spray (active ingredients are, for example, beta-2-sympathomimetics, cortisone, antileukotrienes) usually relieves the symptoms; in the case of very severe attacks, the doctor injects the drug directly into the vein.

Chronic obstructive pulmonary disease (COPD) is a group of diseases of the lungs in which the airways are permanently narrowed and characterized by coughing, increased sputum, and shortness of breath on exertion. It primarily includes chronic obstructive bronchitis and emphysema, which are characterized primarily by difficult exhalation. The narrowing (obstruction) of the airways is usually a result of smoking, but dust, fumes and gases can also cause COPD.

COPD cannot be cured. However, medication can be used to alleviate symptoms, reduce the number of coughing attacks and prevent this lung disease from progressing further. In addition, the physical resilience can be improved, relapses and complications can be prevented and thus the quality of life and life expectancy can be increased.

Further, the pharmaceutical preparation provided in the form of a medicament inhalable into the lungs may preferably be provided for use in the treatment of cystic fibrosis, SIRS (systemic inflammatory response syndrome), sepsis, pulmonary fibrosis, bronchial carcinoma, lung cancer (lung carcinoma), emphysema, pulmonary hypertension (pulmonary hypertension), bronchitis, pneumonia, interstitial lung disease and/or tuberculosis.

The term SIRS (systemic inflammatory response syndrome) is well known and was defined at a conference on sepsis and organ failure in 1991 (Bone, R. C., et al. (1992). “Definitions of Sepsis and Organ Failure and Guidelines for the Use of Innovative Therapies in Sepsis. ACCP/SCCM Consensus Conference Committee. American College of Chest Physicians/Society of Critical Care Medicine.” Chest 101(6): 1644-1655).

SIRS is caused by various triggering events, such as infections with bacteria, fungi, or injuries, but they have in common that a sequence of reactions of the body occurs leading to a life-threatening condition with multiple organ failure and shock (drop in blood pressure). The term sepsis was coined by Schottmüller for the variant of this condition with infectious causes triggered by bacteria, viruses, fungi, or parasites, a few years after Pasteur first detected bacteria in the blood of ill patients in 1880 (Annane, D., et al. (2005). “Septic shock.” Lancet 365(9453): 63-78).

Over 100 different diseases of the lungs can lead to pulmonary fibrosis. In pulmonary fibrosis, connective tissue is increasingly formed between the alveoli and the blood vessels in the lungs as a result of an inflammatory reaction. As a result, less oxygen reaches the blood. The stiffening of the lungs makes breathing more difficult. This leads to a reduction in physical capacity with shortness of breath. Since the causes of this lung disease are not always known, pulmonary fibrosis is divided into diseases with known and with unknown cause (idiopathic pulmonary fibrosis)

Known causes include, for example, inhalation of asbestos or certain organic substances (e.g., protein components of hay dust or components of pigeon droppings). Diagnosis includes computed tomography (CT), pulmonary function tests, and bronchoscopy. Once the cause is known, the first step is to avoid contact with the triggering substance. The inflammation is treated with anti-inflammatory drugs (e.g., cortisone, azathioprine). Depending on the cause, further therapies may be required.

Cancer of the lungs or bronchi is called lung cancer (lung carcinoma) or bronchial cancer. Carcinoma of the lung is the third most common cancer in Germany. Smoking is considered the main risk factor for the development of lung cancer. Cigarette smoking can be assumed to be the main cause in 80 to 90% of male lung cancer patients and in 30 to 60% of female lung cancer patients. Other risk factors are inhaled dusts and vapors at the workplace (e.g., asbestos, quartz dusts, arsenic, chromates, nickel, and aromatic hydrocarbons), environmental influences (e.g., the radioactive noble gas radon, high levels of pollutants in the air), and, to some extent, hereditary predisposition.

Carcinoma of the lung becomes apparent very late; symptoms are often of a general nature, such as cough, shortness of breath or weight loss. If a tumor is suspected in the area of the lung, an X-ray overview is taken. This is often followed by computed tomography and bronchoscopy. In principle, therapy consists of removal of the tumor, chemotherapy or radiation, or a combination of these options.

Pulmonary emphysema is usually considered a form of chronic obstructive pulmonary disease in which the alveoli are irreversibly dilated and destroyed. As the partition walls of the alveoli are enzymatically dissolved, large bubbles form in which the breathing air becomes trapped. Although the lungs contain air, shortness of breath occurs. As a result, the body is not supplied with sufficient oxygen, and damage to organs may occur. Smoking is considered the main cause of emphysema. Other risk factors include polluted indoor air, open fires, inhalation of gases and dusts at the workplace, and possibly genetic predisposition and frequent infections of the respiratory tract.

Inter alia pulmonary emphysema can be diagnosed by pulmonary function tests (e.g. spirometry), blood gas analysis and imaging techniques (e.g. X-ray of the lungs). In addition to stopping smoking immediately or avoiding other triggering stimuli, surgery can be performed to reduce the size of the lung and remove particularly large bubbles; in extreme cases, transplantation of the lung or a lobe of the lung may also be necessary.

In pulmonary hypertension, there is increased blood pressure in the pulmonary circulation, which leads to shortness of breath, reduced oxygen supply to the body and reduced physical performance. In addition, there may be chest pain and edema in the legs. The causes of pulmonary hypertension are not yet fully understood. However, there are indications that this lung disease occurs more frequently in HIV infection, certain autoimmune diseases and certain drugs (e.g. appetite suppressants, psychogenic stimulants).

A genetic predisposition is also discussed. Based on the symptoms, cardiac output measurements (ECG), X-ray of the thorax and pulmonary function tests are often performed. But it is transthoracic echocardiography, an ultrasound examination from outside through the chest, that provides clues to pulmonary hypertension. Blood pressure in the pulmonary circulation is measured by a specialist using a heart-lung catheter (right heart catheter). Pulmonary hypertension is primarily treated with medication.

Bronchitis is the inflammation of the mucous membrane in the bronchi of the lungs. It can be acute or chronic. Chronic bronchitis is the form of bronchitis in which cough and sputum occur on most days for at least three months in two consecutive years. The cause of chronic bronchitis is not pathogens, but cigarette smoke (or its ingredients) or other inhaled irritants. In contrast, a newly developed inflammation of the bronchial mucosa with cough, mucus production, fever and other non-specific symptoms is called acute bronchitis. The triggers of this lung disease are usually viruses, in rare cases also bacteria. Therefore, acute bronchitis usually heals without drug treatment, only in case of bacterial cause an antibiotic is effective. To prevent chronic bronchitis from developing into chronic obstructive bronchitis or even emphysema, the patient must take care to avoid the triggering irritants (dusts, gases or vapors). There are some medicines that alleviate the symptoms of chronic bronchitis.

Pneumonia is an acute or chronic inflammation of the lung tissue, usually caused by an infection with bacteria (mostly Streptococcus pneumoniae), viruses or fungi. The inflammation can affect the alveoli, the lung tissue between the alveoli, or the blood vessels. Particularly at risk are the elderly, infants and young children, and immunocompromised individuals in whom the immune system is either not yet fully developed or has only limited functionality. The suspected diagnosis can often already be made after physical examination and on the basis of the clinical picture. In most cases, an X-ray of the lungs is taken for confirmation or a sample of the sputum is taken to determine the causative agent. As a rule, treatment is with an antibiotic.

The interstitial lung diseases comprise various diseases of the lung and occur rather rarely. They affect the intermediate tissue (interstitium) of the lung or the alveoli (air sacs). Interstitial lung diseases include, for example, pulmonary fibrosis (pathological proliferation and scarring of the lung tissue) or sarcoidosis (formation of small connective tissue nodules in the lung).

Tuberculosis is an infectious disease caused by bacteria, which mainly affects the lungs. In the majority of people infected with tuberculosis, the disease is not noticeable.

The pharmaceutical preparation is characterized by very good tolerability and low side effects. In this regard, the reactive chlorine compound may be combined with other treatment methods and/or further active ingredients to improve patient survival. Preferably, the treatment of a lung disease in a human individual may comprise simultaneous or sequential administration to the individual of cystic fibrosis therapy, exercise therapy, nutritional therapy, and/or blood washing therapy.

Another object of the present invention is a use of a reactive chlorine-oxygen compound for the manufacture of a medicament for the prophylactic and/or therapeutic treatment of a lung disease.

Another object of the present invention is a combination preparation comprising separate packages of at least one pharmaceutical preparation for use in the form of a medicament inhalable into the lungs and/or for the treatment of a lung disease according to the present invention and at least one medicament different from the previously set forth reactive chlorine-oxygen compound as it is the subject of the present application.

Preferred further medicaments different from a reactive chlorine compound include, but are not limited to, antibiotics, antipyretics, drugs for the treatment of Disseminated Intravascular Coagulopathy (DIC), antibodies, cytokines, chemokines, antimicrobial peptides, sphingomyelinase inhibitors, statins, alpha-2-macroglobulin, thrombin-derived C-terminal peptide, sphingosine-1-phosphate, curcumin, ascorbic acid, resveratrol, melatonin, glycyrrhizin, and erythropoietin, Steroids, such as cortisone; beta-2 sympathomimetics, such as indacaterol, salbutamol, fenoterol, reproterol, salmeterol, and formoterol; anticholinergics, such as butylscopolamine, ipratropium bromide, and tiotropium bromide. These active ingredients can be used singly or as a mixture of two, three, four, or more.

Preferred antibiotics include inter alia β-lactam antibiotics, such as penicillins, in particular benzylpenicillin, phenoxymethylpenicillin, propicillin, azidocillin, flucloxacillin, dicloxacillin, cloxacillin, oxacillin, methicillin, amino penicillins, such as amoxicillin, ampicillin and bacampicillin, acylamino penicillins, such as mezlocillin and piperacillin, pivmecillinam; cephalosporine, basiscephalosporine, cefuroxim, cefamandol, cefoxitin, cefotiam, cefotaxim, cefovecin, ceftazidim, cefepim, cefodizim, ceftriaxon; oralcephalosporine, cefaclor, cefadroxil, cefalexin, loracarbef, cefixim, cefuroximaxetil, cefetametpivoxil, ceftibuten, cefpodoximproxetil; β-lactamase inhibitors, in particular sulbactam, clavulan acid in combination with amoxicillin, tazobactam in combination with piperacillin, carbapeneme, imipenem in combination with cilastatin, meropenem, doripenem, ertapenem, monobactame, such as aztreonam.

Furthermore, preferred antibiotics include glycopeptides, such as vancomycin, dalbavancin and teicoplanin.

Preferred antipyretics include inter alia non-steroidal antirheumatic drugs, such as ibuprofen, naproxen, nimesulide and ketoprofen (arylpropionic acid derivatives), acetylsalicylic acid; paracetamol (aminophenol derivatives); pyrazolone derivates, such as phenazone, propyphenazone, metamizole; nabumetone (arylacetic acid derivatives) and quinine.

Preferred drugs for the treatment of disseminated intravascular coagulation (DIC) include antithrombin, protein C and APC (activated protein C), thrombomodulin (TM), heparin, tissue factor pathway inhibitor (TFPI).

Preferred antibodies include inter alia immunoglobulins, cytokine inhibitors, in particular antibodies against IL-1, IL-17A or IL-18; antibodies or other molecules, which block the binding of PD-L1 to its receptor PD-1 (programmed cell death-1); and antibodies and other blocking molecules directed against surface proteins on thrombocytes (abciximab, tirofiban, and eptifibatide) and immune cells (CD39).

Preferred cytokines include inter alia interleukin 7(IL-7), IL-15 and GM-CSF (granulocyte-macrophage colony stimulating factor).

Preferred chemokines include inter alia CXCL10.

Preferred antimicrobial peptides include inter alia thymosin alpha 1.

Preferred sphingomyelinase inhibitors include inter alia amitriptyline.

Preferred statins include inter alia atorvastatin and simvastatin.

Another object of the present invention is an inhaler with a pharmaceutical preparation to be nebulized, characterized in that the pharmaceutical preparation comprises at least one reactive chlorine-oxygen compound.

Inhalers are medical devices used to generate aerosols or vapors, wherein the generated aerosols or vapors can be inhaled by patients. Thus, inhalers generally comprise a container of a pharmaceutical preparation and a means for generating an aerosol or a vapor from the pharmaceutical preparation present in the container and a means for facilitating inhalation of the generated aerosol or vapor by the patient. The means for generating an aerosol or a vapor from the pharmaceutical preparation present in the container may be, for example, a pressurizing device, in particular a compressor, which provides an air flow that is passed through a nebulizer. In the nebulizer, an aerosol or vapor is generated from the pharmaceutical preparation present therein, which via a tube directs the aerosol or vapor into a mouthpiece or respiratory mask that facilitates the patient's inhalation of the generated aerosol or vapor.

In a preferred embodiment, it can be provided that the inhaler provides a volumetric flow rate in the range of 0.05 l/s to 10 l/s, preferably from 0.1 l/s to 4 l/s, particularly preferably 0.2 l/s to 3 l/s (liters per second). It can further be provided that the inhaler provides a nebulization time of at most 10 min for 2.5 ml of pharmaceutical preparation. It can further be provided that the inhaler provides an aerosol with an average particle size of at most 10 μm, preferably at most 5 μm, more preferably at most 3.5 μm, measured as Mass Median Aerodynamic Diameter (MMAD) with a 7-stage cascade impactor (Next Generation Impactor (NGI)) (DIN EN 13544-1:2007+A1:2009).

Inhalers with these properties are widely known and commercially available. These include, among others, the “MicroDrop® Pro2” device from MPVMedical, the “Inhaler IH 26” device from Beurer, the “IN 550 Inhaler” device from Medisana, the “InnoSpire Elegance” device from Philips, the “CompAir NE-028P” device from Omron Healthcare, and the “BR-CN 116” device from Omnibus. Furthermore, such devices are described, among others, in the publications DE 20 2009 004147 U1 and DE 10 2004 049338 A1.

The following examples explain the invention in more detail, but are not in any way restrictive.

The efficacy of reactive chlorine compounds, preferably peroxochloric acids and dichloroxo acid, particularly preferably dichloroxo acid, can be tested in animal models, for example with regard to antiviral properties

For this purpose, the nebulization of 5 mM DPOCL solution can first be compared to conventional inhalation solutions such as 0.9% NaCl solution (B. Braun, Melsungen) and ISOMAR, sea salt solution 3% (MPV Medical, Putzbrunn) by nebulization with an inhaler, such as a MicroDrop Prop2 inhalation device. The measured nebulization times of all 3 substances under different conditions and with different volumes can all be adjusted within a comparable range with a variance of the nebulization rates of +/−5%. Particularly preferred reactive chlorine compounds, which have a high water content as a carrier, exhibit essentially the same nebulization properties as the salt solutions mentioned above

In suitable and well-established animal models, in which animals are exposed to active substances either in controlled cages or via appropriate masks by inhalation, the effect of reactive chlorine compounds, preferably peroxochloric acids and dichloroxo acids, particularly preferably dichloroxo acids on the inhibition of virally induced lung diseases or other diseases can be tested. For example, in a recent HACE2 model with transgenic mice, the inhibitory effect of dichloroxo acids (especially DPOCI, which is detailed below) can be demonstrated in the infection of mice with coronavirus (COVID-19) (Bao, L.; Deng, W.; Huang, B. et al. The Pathogenicity of SARS-CoV-2 in hACE2 Transgenic Mice. Nature 2020)

FIG. 1 shows the titration of the anions of peroxo acids (dichloric acid, peroxochlorous acid) present in the solution to determine the concentration of acid anions.

FIG. 2 shows the derivation of the titration curve of FIG. 1, which is used for the exact determination of the concentration.

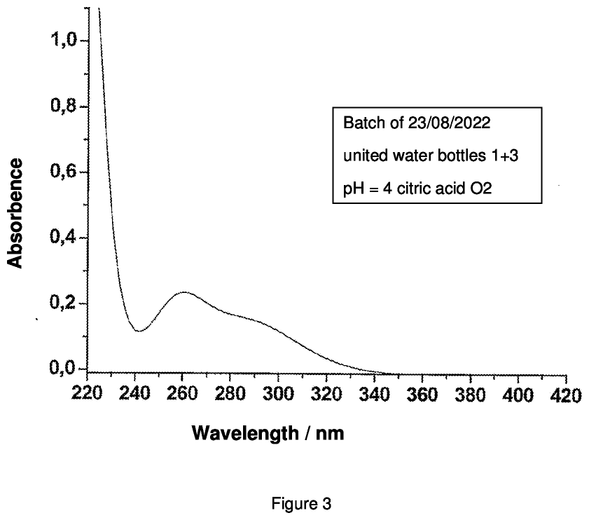

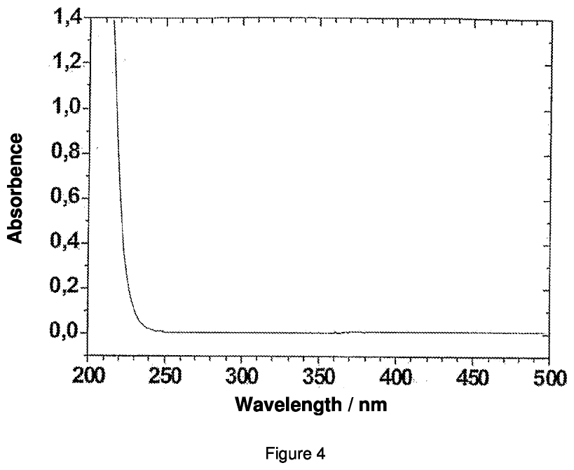

FIGS. 3 and 4 show examples of UV spectra. The UV absorption measurements allow the concentration of existing chlorite to be determined and indicate any dissolved free chlorine dioxide present.

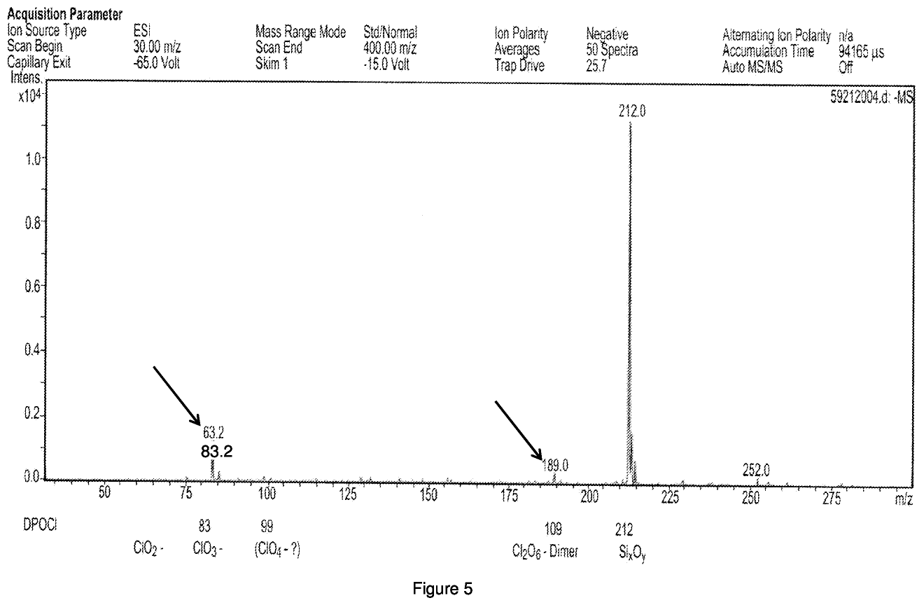

FIG. 5 shows a mass spectrum of the product solution, wherein the peroxochlorite (mass 83.2) and the anion of dichloric acid (mass 189) were detected.

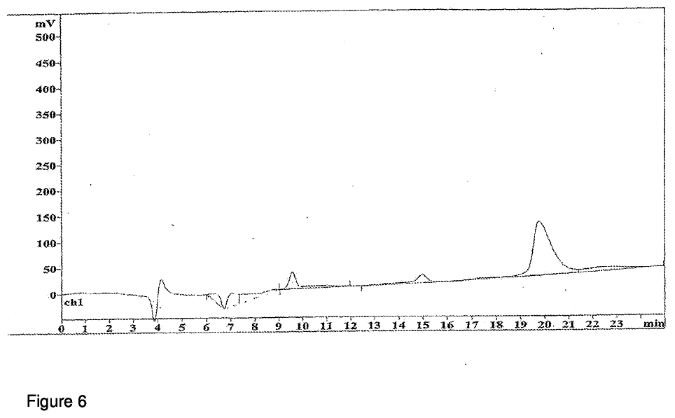

FIG. 6 shows the result of ion chromatography. The retention times of comparative substances are given in example 4, part 5. The dichloric acid is detected at 19.77 min, and no chlorate (ClO₃⁻) is detectable, which rules out chlorate as the cause of the peak in the mass spectrum at 82.3 in FIG. 5.

### EXAMPLE 1

### Preparation of Dichloric Acids

Drops of sulfuric acid (96%) are added carefully while stirring to a solution of 100 g anhydrous sodium chlorite in 200 mL water. With a strong gas flow (Ar, N₂ or O₂ or CO₂-free air) the chlorine dioxide produced is expelled. The gas flow has to be so strong that the ClO₂ content does not rise above 5 percent (risk of explosion). The 0102-containing gas flow, is introduced into a solution of 15 mL 30% hydrogen peroxide in 35 mL water, which has previously been brought to pH 12 by the addition of 4M sodium hydroxide solution, via three washing bottles connected in series, each of which is filled with 30 mL of a 2 M NaClO₂ solution with pH 11, in order to capture elemental chlorine. Instead of hydrogen peroxide also a solution of sodium perborate or sodium percarbonate or another peroxo compound can be used such as the H₂O₂ adduct of urea. During the introduction of gas the pH is checked by a glass electrode. By adding 4M NaOH, the pH is kept at 12 for the course of the reaction. The supplied hydroperoxide or supplied peroxo compound is used up when the introduction of gas leads to a permanent yellow coloration. The yellow solution is then decolored again with a drop of the solution of the oxidizing agent (e.g. H₂O₂).

The solution containing reactive chlorine is dropped while stirring to a solution of 500 g citric acid in 3 liters of water, which has been previously adjusted to pH 4.5 with 2 M sodium hydroxide solution. During the addition, the reactive chlorine compound formed is expelled by a powerful gas flow (N₂ or O₂). The gas flow should preferably be cooled. The hose connections should be as short as possible. The gas is collected for example in three wash bottles connected in series, each charged with 50 mL 0.1 M NaOH.

The contents of the wash bottles are combined and kept at pH >10.

For forming the dichloric acids which are preferably used according to the invention the pH is adjusted to 7 for example with hydrochloric acid and a 10-fold molar excess amount of sodium chlorite is added.

In a further embodiment, the pH is adjusted to 7 for example with hydrochloric acid and an equimolar amount of sodium chlorite is added for the formation of the dichloric acids which are preferably used according to the invention.

For storage, it is then preferred in each case if the pH is adjusted to approximately 10 to 13.

The total content of reactive chlorine anions is determined by potentiometric titration with 0.1 M HCl in a manner well known to the person skilled in the art. Here, different compounds can be determined on the basis of the pKa values of the different anions obtained over the titration curve.

The dichloric acids formed are present in solution in a mixture with a defined amount of chlorite and other reactive chlorine compounds.

The presence of the dichloric acids is detected by Raman spectroscopy.

### EXAMPLE 2

### Analytical Determinations of the Solution Obtained from Example 1

### 1) pH Measurement:

The pH is determined by the single rod glass electrode. The product content and the state of equilibrium are dependent on the pH value.

2) Titration with 0.1 M HCl:

The titration is used for example for quantitatively determining the dichloric acid content or also the content of peroxochlorous acid or peroxochlorate.

1 mL of the product solution is titrated potentiometrically with 0.1 M hydrochloric acid. Titration curves (pH vs. mL 0.1 M HCl) are recorded. From the acid consumption between pH 8.5 and 4.5 determined in the derivation of the titration curve, the content of anions of the corresponding acids in total is determined.

In a typical result 1 mL product solution gives a consumption of 0.72 mL 0.1 M HCl and thus a concentration of 0.072 M.

FIG. 1 shows a recorded titration curve.

FIG. 2 shows the derivation of the titration curve and determination of the concentration.

### 3) UV-Vis Absorption Spectrum:

The measurement of the UV spectrum is used to quantify the content of chlorite in the product solution. For comparison, spectra of a chlorite-containing and a chlorite-free product solution are shown in FIGS. 3 and 4 respectively. The chlorite signal is at 260 nm; chlorine dioxide from the process shows a signal at 360 nm.

In 1 cm quartz cuvettes the absorbance values are determined at 260 nm and 500 nm. From the difference A260-A500 and by means of the extinction coefficient for chlorite of ε260 nm=140 M⁻¹ cm⁻¹ at 260 nm, the content of ClO₂⁻ ions can be determined.

Absorption at 360 nm indicates free chlorine dioxide (ε360 nm=1260 M⁻¹ cm⁻¹).

### 4) Mass Spectroscopy

The ESI mass spectrometry was performed with a Bruker Esquire-LC spectrometer in standard MS mode. The sample was an aqueous product solution diluted with methanol before measurement. The scan range used was between 30 m/z and 400 m/z, with capillary exit—65 volts and skim—15 volts; the spectrum represents an average of 50 measurements.

The right arrow in FIG. 5 indicates the signal of the dichloric acid (molecular formula: Cl₂O₆²⁻), the left arrow shows the signal of the peroxochlorite species (molecular formula: ClO₃⁻).

### 5) Ion Chromatography

All analyses were carried out with a modular ion chromatography system of the company Metrohm.
Pump: Metrohm IC 709 Pump
Detector: Metrohm 732 IC Detector
Suppressor: Metrohm 753 Suppressor Module
Column: Metrosep A 250
Flow rate: 1 ml/min
Injection volume: 20 μL
Eluent: 1 mM NaOH

Fresh solutions of reference substances of known concentration were prepared immediately before each measurement and then measured using the method described above with the specified eluent.

### Retention Times of the Reference Substances:

| Substance | Retention time [min] |
|---|---|
| NaCl | 13.21 |
| NaClO₂ | 12.30 |
| NaClO₃ | 16.26 |
| NaClO₄ | 4.36 |
| NaOH | 17.32 |
| Na₂CO₃ | 21.98 |
| Na₂Cl₂O₆ | 19.77 |

FIG. 6: in ion chromatography, dichloric acid yields a typical peak at a retention time of 19.77 min. None of the known reference substances could be detected. The ion chromatography confirms the observation from mass spectroscopy. A chlorate-typical peak (NaClO₃, retention time 16.26 min) is not detectable in the solution prepared according to Example 1. Therefore, the peak of the molecular formula ClO₃⁻ in the mass spectroscopy (FIG. 5, mass 83.2) can only be the dichloroperoxo acid or its ion, which is particularly preferable to use.

### EXAMPLE 3

Experiments with viruses to demonstrate the effectiveness of reactive chlorine compounds to be used according to the invention. A solution of reactive chlorine compounds prepared according to Example 1 and analyzed in more detail according to Example 2 is abbreviated below as DPOCL.

### Material

DPOCL solution 5 mM
Loading substance 3.0 g/l % BSA or 3.0 g/l BSA+3.0 ml/l Erys
Virus Modified Vaccinia virus (MVA), Strain Ankara, Passage 4, ZK-1608

### Test

In a test based on DIN 14476, 8 parts of test substance are mixed with one part each of loading substance and virus suspension:
1 ml of loading substance is pipetted into a container whose volume is suitable for sufficient mixing. 1 ml of virus test suspension is added to the container, taking care not to touch the upper part of the inside. It is mixed well.

8 ml of product test solution (test substance) is added to the container. It is mixed, the stopwatch is started immediately and the container is placed in a water bath set to the selected test temperature (20+/−1° C.) in a controlled manner for 30 or 90 minutes. The effect of the product is determined for the selected exposure times. Immediately after the selected exposure time has elapsed, mix thoroughly and pipette 0.5 ml of the test mixture into 4.5 ml of ice-cooled MEM and place in an ice bath. Within 30 min, a dilution series with a factor of ten is prepared from this mixture (test mixture+MEM). The pipette tips must be changed after each dilution to prevent virus carryover. After incubation, calculate the virus titer; the reduction in virus infectivity is calculated from the difference in lg virus titer before and after treatment with the product.

In the test performed, the total volume was reduced to 1 mL and the dilution series was not performed with a factor of 10, but with a factor of 3, since more accurate results are obtained in this way.

### Result

Under the selected conditions at 30 and 90 minutes incubation time and a temperature of 20+/−1° C., the loading substance 3.0 g/l % BSA shows an average reduction of the virus titer of more than 90%. The tests with the loading substance 3.0 g/l BSA+3.0 ml/l Erys also lead to comparable results.

### EXAMPLE 4

Experiments with viruses to demonstrate the effectiveness of reactive chlorine compounds to be used according to the invention. A solution of reactive chlorine compounds prepared according to Example 1 and analyzed in more detail according to Example 2 is abbreviated below as DPOCL.

Human CaCo-2 cells from colon carcinoma obtained from the German Collection of Microorganisms and Cell Cultures (DSMZ; Braunschweig, Germany) are used for the assay. Cells are cultured at 37° C. in MEM (Minimal Essential Medium) containing 10% FCS (fetal calf serum) with 100 IU/ml penicillin and 100 μg/ml streptomycin. All chemicals are purchased from standard suppliers such as Sigma.

SARS-CoV-2 viruses are prepared from samples of travelers returning from Wuhan (China) to Frankfurt am Main (Germany). The viruses are propagated in the human CaCo-2 cell line from colon carcinoma. SARS-CoV-2 viruses used for testing are cultured one passage in the CaCo-2 cells and frozen at −80° C. for storage. Virus titer was determined as TCID50/ml in confluent cells in 96-lock microtiter plates. (The TCID50—Median Tissue Culture Infectious Dose) is a method for checking viral titer. TCID50 denotes the concentration at which 50% of the cells are infected.

To test the antiviral properties of DPOCI, confluent cell races of CaCo-2 cells in 96-hole microtiter plates are treated with a suspension of SARS-CoV-2 virus at a MOI (multiplicity of infection; virus particle to cell ratio) of 0.01. The virus suspension is added simultaneously with different concentrations of DPOCI in MEM culture medium containing 2% FCS. The cytopathogenic effect (CPE) is examined 48 hours after infection has occurred. To investigate the effect of DPOCI on the viability of CaCo-2 cells, confluent cell lawns are exposed to different test conditions and concentrations. Cell viability will be determined using a typical viability assay e.g.: Rotitest Vital (Roth) or the MTT assay. At least one triplicate determination is performed in each case and the results are presented in the form of dose-response curves.

The features of the invention disclosed in the above description, as well as in the claims, figures and embodiments, may be essential, both individually and in any combination, for the implementation of the invention in its various embodiments.

1. A pharmaceutical preparation containing, as an active ingredient, a reactive chlorine-oxygen compound, wherein the reactive chlorine-oxygen compound is provided in the form of a medicament inhalable into a patient's lungs.

2. The pharmaceutical preparation according to claim 1, wherein reactive chlorine-oxygen compound is provided as an aerosol.

3. The pharmaceutical preparation according to claim 1, wherein the reactive chlorine-oxygen compound is provided as an aerosol with an average particle size of at most 10 μm, measured as Mass Median Aerodynamic Diameter (MMAD) with a 7-stage cascade impactor (Next Generation Impactor (NGI)) (DIN EN 13544-1:2007+A1:2009).

4. The pharmaceutical preparation according to claim 1, wherein the reactive chlorine-oxygen compound comprises a peroxochloric acid, a dichloroperoxo acid, and/or a peroxochlorous acid or a pharmaceutically acceptable salt of these acids.

5. The pharmaceutical preparation according to claim 1, wherein the reactive chlorine-oxygen compound comprises a molecular formula selected from HClO₃, HClO₄, and/or H₂Cl₂O₆ or a pharmaceutically acceptable salt of these acids.

6. The pharmaceutical preparation according to claim 1, wherein the reactive chlorine-oxygen compound comprises a structure of formula [O═ClOO]⁻, [O₂ClOO]⁻, [O₂ClOOClO₂]²⁻, and/or an anion of the reactive chlorine-oxygen compound has a molecular formula Cl₂O₆²⁻.

7. The pharmaceutical preparation according to claim 1, wherein the reactive chlorine-oxygen compound is obtainable according to a method in which
(a) chlorine dioxide is reacted with an aqueous or water-containing solution of hydrogen peroxide or another hydroperoxide or peroxide at a pH>=6.5,
(b) the pH is lowered to 3 to 6 by the addition of an acid, and
(c) the gaseous free reactive chlorine-oxygen compound is expelled with a cooled gas and collected in a basic solution with a pH>10.

8. The pharmaceutical preparation according to claim 1, wherein the reactive chlorine-oxygen compound is obtainable according to a method in which
(a) chlorine dioxide is reacted with an aqueous or water-containing solution of hydrogen peroxide or another hydroperoxide or peroxide at a pH>=6.5,
(b) the pH is lowered to 3 to 6 by the addition of an acid,
(c) the gaseous free reactive chlorine-oxygen compound is expelled with a cooled gas and collected in a basic solution with a pH>10, and
(d) the collected reactive chlorine-oxygen compound is incubated with chlorite at a pH of 6 to 8.

9. The pharmaceutical preparation according to claim 1, wherein a pharmaceutically acceptable carrier is used.

10. The pharmaceutical preparation according to claim 1, wherein a mass spectrum of the pharmaceutical preparation shows a signal at 189.0 m/z.

11. The pharmaceutical preparation according to claim 1, wherein the pharmaceutical preparation is provided for use in treatment of a lung disease.

12. The pharmaceutical preparation according to claim 11, wherein the lung disease is a mycotic, bacterial, or viral infection.

13. The pharmaceutical preparation claim 1, wherein the pharmaceutical preparation is provided for use in treatment of a chronic lung disease.

14. The pharmaceutical preparation according to claim 11, wherein the chronic lung disease is bronchial asthma and/or COPD (chronic obstructive pulmonary disease).

15. The pharmaceutical preparation according to claim 1, wherein the pharmaceutical preparation is provided via an inhaler.

16. A use of a reactive chlorine-oxygen compound for the manufacture of a medicament for prophylactic and/or therapeutic treatment of a lung disease.

17. A combination preparation comprising separate packages of at least one pharmaceutical preparation according to claim 1 for treatment of a lung disease and at least one medicament which differs from the reactive chlorine-oxygen compound of claim 1.

18. An inhaler with a pharmaceutical preparation to be nebulized, wherein the pharmaceutical preparation comprises at least one reactive chlorine-oxygen compound.

19. The pharmaceutical preparation according to claim 3, wherein the reactive chlorine-oxygen compound is provided as an aerosol with an average particle size of at most 5 μm, measured as Mass Median Aerodynamic Diameter (MMAD) with a 7-stage cascade impactor (Next Generation Impactor (NGI)) (DIN EN 13544-1:2007+A1:2009).

20. The pharmaceutical preparation according to claim 19, wherein the reactive chlorine-oxygen compound is provided as an aerosol with an average particle size of at most 3.5 μm, measured as Mass Median Aerodynamic Diameter (MMAD) with a 7-stage cascade impactor (Next Generation Impactor (NGI)) (DIN EN 13544-1:2007+A1:2009).

21. The pharmaceutical preparation according to claim 8, wherein the reactive chlorine-oxygen compound is obtainable according to a method in which in the collected reactive chlorine-oxygen compound is incubated with chlorite at a pH of approximately 7.

22. The pharmaceutical preparation according to claim 9, wherein the pharmaceutically acceptable carrier comprises water.

23. The pharmaceutical preparation according to claim 22, wherein the water content of the pharmaceutically acceptable carrier is at least 90 wt.-%.

24. The pharmaceutical preparation according to claim 23, wherein the pharmaceutical preparation is an aqueous solution.

25. The pharmaceutical preparation according to claim 12, wherein the lung disease is a bacterial or viral infection.

26. The pharmaceutical preparation according to claim 25, wherein the lung disease is a viral infection.
